(19) United States
(12) Patent Application Publication
(10) Pub. No.: US 2011/0092826 A1
(43) Pub. Date: Apr. 21, 2011

(54) System and method for measuring ECG and breath signals by using two polar electrodes

(75) Inventors: Chao-Fa Lee, Taipei (TW); Shu-Ting Liao, Taipei (TW); Cheng-Hsing Kuo, Taipei (TW)

(73) Assignee: Tatung Company, Taipei (TW)

(21) Appl. No.: 12/591,862

(22) Filed: Dec. 3, 2009

(30) Foreign Application Priority Data
Oct. 20, 2009 (TW) 098135392

(51) Int. Cl. A61B 5/0205

(52) U.S. Cl. 600/484

(57) ABSTRACT

A system for measuring ECG and breath signals by using two polar electrodes includes a first polar electrode, a second polar electrode and a breath and ECG signals measuring device. The breath and ECG signals measuring device includes a level regulator, a square wave generator, a breath signal process module and an ECG signal process module. The first and second polar electrodes are attached on the body of a person under test to import square wave signal and receive the first and second measuring signals. The level regulator adjusts the voltage reference of the first and second measuring signals. The breath signal process module and the ECG signal process module process and generate a breath signal and an ECG signal respectively.

### BACKGROUND OF THE INVENTION

1. Field of the Invention

The present invention relates to the technical field of measuring ECG and breath signals and, more particularly, to a system and method for measuring ECG and breath signals by using two polar electrodes.

2. Description of Related Art

Heart of human beings is composed of heart muscles, and the change in potential of the heart would reflect to the surface of body through conductive tissues and liquid humors around the heart when heart muscles contract to expel blood for delivering to the body. Thus, the method for measuring ECG (Electrocardiogram) in the prior art is typically to attach two polar electrodes on two sides of the body of the person under test so as to record the difference between the internal and external potential of heart muscle cells generated by the variation of tiny electrical pulses of the heart, and the ECG signal of the person under test is obtained by amplifying and filtering the difference.

The method for measuring breath signal in the prior art is to attach two polar electrodes on the surface of the body of the person under test so as to import high-frequency current, and preferably the two polar electrodes are attached on the left and right hands or beside of the heart respectively. Since the thoracic impedance change caused by breathing may decrease or increase the imported high-frequency current, the breath signal could be obtained by measuring the peak-to-peak value of the changed current passing through the two polar electrodes. If a system with two polar electrodes is used to measure the ECG and breath signals at the same time, technically, the high-frequency current for measuring the breath signal would interfere with the ECG signal, and a ground-free ECG system also is higher electrical interference by imbalance between the active electrodes. To solve the problem above, the method in the prior art is to additionally provide a grounding polar electrode for use as a reference, and hence three polar electrodes should be attached on the body of the person under test, which is not convenient in use. Therefore, it is desirable to provide an improved measuring system and method to mitigate and/or obviate the aforementioned problems.

### SUMMARY OF THE INVENTION

The object of the present invention is to provide a system and method for measuring the breath and ECG signals simultaneously by using two polar electrodes so as to attach only two polar electrodes on the left and right hands or beside of the heart of the person under test for measuring the ECG and breath signals.

According to a feature of the invention, the invention provides a system for measuring ECG and breath signals by using two polar electrodes, which comprises: a first polar electrode attached on a first specified part of a person under test for importing electric current and sensing a first measuring signal; a second polar electrode attached on a second specified part of the person under test for importing electric current and sensing a second measuring signal; and a breath and ECG signals measuring device connected to the first polar electrode and the second polar electrode, the breath and ECG signal measuring device including a level regulator, a square wave generator, a breath signal process module, and an ECG signal process module, wherein the breath signal process module and the ECG signal process module are connected to the level regulator and the square wave generator respectively, the level regulator adjusts the voltage reference of the first and second measuring signals, the square wave generator generates square signals with a fixed frequency, the breath signal process module receives the first and second measuring signals and proceeds to generate a breath signal, the ECG signal process module receives the first and second measuring signals and proceeds to generate an ECG signal.

According to another feature of the invention, the invention provides a method for measuring ECG and breath signals by using two polar electrodes, which is applied in a system including a first polar electrode, a second polar electrode, and a breath and ECG signal measuring device connected to the first and second polar electrodes, the first polar electrode importing electric current and sensing a first measuring signal, the second polar electrode importing electric current and sensing a second measuring signal, the breath and ECG signal measuring device including a level regulator, a square wave generator, a breath signal process module, and an ECG signal process module, the level regulator adjusting the voltage reference between the two measuring signals, the square wave generator generates square signals, the breath signal process module receiving the first and second measuring signals and proceeding to generate a breath signal, the ECG signal process module receiving the first and second measuring signals and proceeding to generate an ECG signal. The method comprises the steps of: (A) attaching the first and second polar electrodes on a first specified part and a second specified part of a person under test respectively; (B) the square wave generator generating square wave signals with a fixed frequency for being imported into skin of the person under test through the first and second polar electrodes respectively; (C) the first and second polar electrodes receiving the first and second measuring signals respectively; (D) the level regulator adjusting levels of the first and second measuring signals respectively; (E) the breath signal process module executing for the first and second measuring signals to generate a breath signal; (F) the ECG signal process module processing the first and second measuring signals to generate an ECG signal; and (G) outputting the ECG and breath signals.

Other objects, advantages, and novel features of the invention will become more apparent from the following detailed description when taken in conjunction with the accompanying drawings.

### BRIEF DESCRIPTION OF THE DRAWINGS

FIG. 1 is a schematic diagram of the system for measuring the ECG and breath signals by using two polar electrodes in accordance with the present invention;

FIG. 2 is a flowchart of the method for measuring the ECG and breath signals by using two polar electrodes in accordance with the present invention;

FIG. 3 is a schematic diagram of the peripheral circuit for the two polar electrodes of the system for measuring the ECG and breath signals by using two polar electrodes in accordance with one preferred embodiment of the present invention;

FIG. 4 is a circuit diagram of the ECG signal process module of the system for measuring the ECG and breath signals by using two polar electrodes in accordance with one preferred embodiment of the present invention;

FIG. 5 is a circuit diagram of the breath signal process module of the system for measuring the ECG and breath signals by using two polar electrodes in accordance with one preferred embodiment of the present invention.

### DETAILED DESCRIPTION OF THE PREFERRED EMBODIMENT

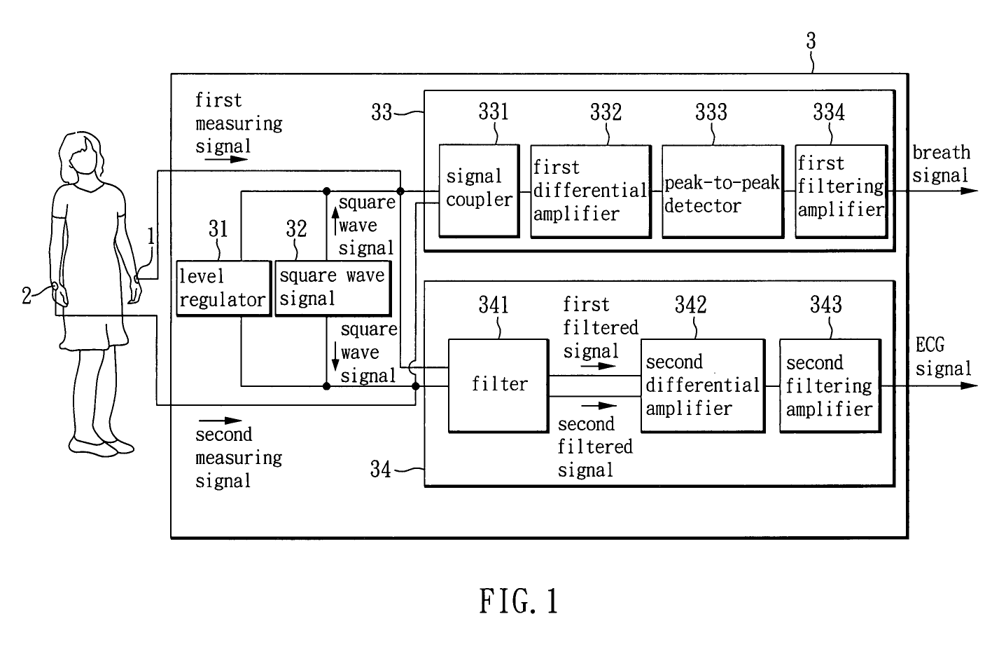

Please refer to FIG. 1. FIG. 1 is a schematic diagram of the system for measuring the ECG and breath signals by using two polar electrodes in accordance with the present invention. The system comprises a first polar electrode 1, a second polar electrode 2, and a breath and ECG signal measuring device 3. The first polar electrode 1 and the second polar electrode 2 are attached on a first specified part and a second specified part of the body of a person under test respectively. Preferably, the first polar electrode 1 and the second polar electrode 2 are attached on the left and right hands or beside of the heart of the person under test respectively, for importing the electric current into the body of the person under test, and sensing a first measuring signal and a second measuring signal respectively.

The aforementioned breath and ECG signal measuring device 3 is connected to the first polar electrode 1 and the second polar electrode 2 respectively. The breath and ECG signal measuring device 3 comprises a level regulator 31, a square wave generator 32, a breath signal process module 33, and an ECG signal process module 34. The breath signal process module 33 and the ECG signal process module 34 are connected to the level regulator 31 and the square wave generator 32 respectively. The level regulator 31 is used for adjusting the level of signals, the square wave generator 32 is provided for generating square signals with a fixed frequency.

The breath signal process module 33 is used for receiving the first and second measuring signals and proceeding to generate a breath signal. The breath signal process module 33 comprises a signal coupler 331, a first differential amplifier 332, a peak-to-peak detector 333, and a first filtering amplifier 334. The signal coupler 331 is connected to the first differential amplifier 332. The peak-to-peak detector 333 is connected to the first differential amplifier 332 and the first filtering amplifier 334. The signal coupler 331 is provided for coupling the first and second measuring signals received by the breath signal process module 33. The first differential amplifier 332 is provided for measuring and amplifying the difference between the first and second measuring signals. The peak-to-peak detector 333 is provided for detecting the peak-to-peak value of the signals from the first differential amplifier 332. The first filtering amplifier 334 is provided for filtering out and rejecting the noises in the signal from the peak-to-peak detector 333 and amplifying the same to generate the breath signal.

The ECG signal process module 34 is used for receiving the first and second measuring signals and proceeding to generate an ECG signal. The ECG signal process module 34 comprises a filter 341, a second differential amplifier 342, and a second filtering amplifier 343. The second differential amplifier 342 is connected to the filter 341 and the second filtering amplifier 343 respectively. The filter 341 is used for filtering out and rejecting the high-frequency signals and noises in the first and second measuring signals received by the ECG signal process module 34 so as to generate first and second filtered signals. The second differential amplifier 342 is used for measuring and amplifying the difference between the first and second filtered signals. The second filtering amplifier 343 is used for filtering out noises in the signals from the second differential amplifier 342 and amplifying the same to generate the ECG signal.

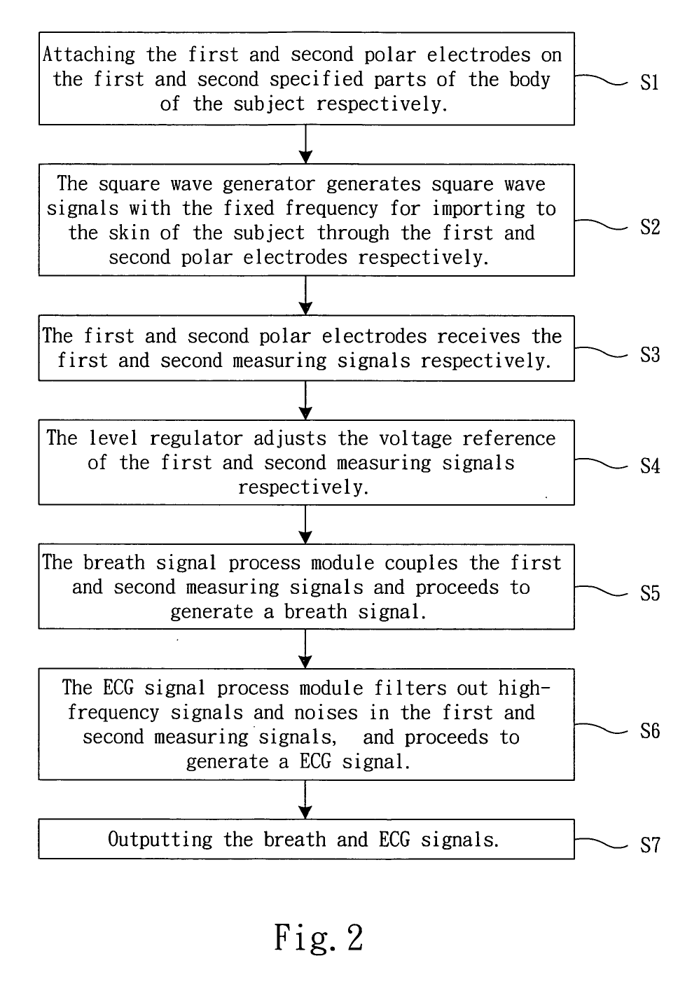

Please refer to FIG. 2. FIG. 2 is a flowchart of the method for measuring the ECG and breath signals by using two polar electrodes in accordance with the present invention. First, the first polar electrode 1 and the second polar electrode 2 are attached on the first and second specified parts of the body of the person under test respectively (step S1). The square wave generator 32 generates square wave signals with the fixed frequency, which is preferably between 12 KHz and 60 KHz, and square wave signals are imported to the skin of the person under test through the first polar electrode 1 and the second polar electrode 2 respectively (step S2). The first polar electrode 1 and the second polar electrode 2 receive the first and second measuring signals respectively (step S3), and sends the same to the breath and ECG signal measuring device 3. The level regulator 31 adjusts the voltage reference of the first and second measuring signals (step S4). The breath signal process module 33 couples the first and second measuring signals and proceeds to generate the breath signal (step S5). The ECG signal process module 34 filters out and rejects the high-frequency signals and noises in the first and second measuring signals and proceeds to generate the ECG signal (step S6). The breath and ECG signal measuring device 3 outputs the breath and ECG signals (step S7).

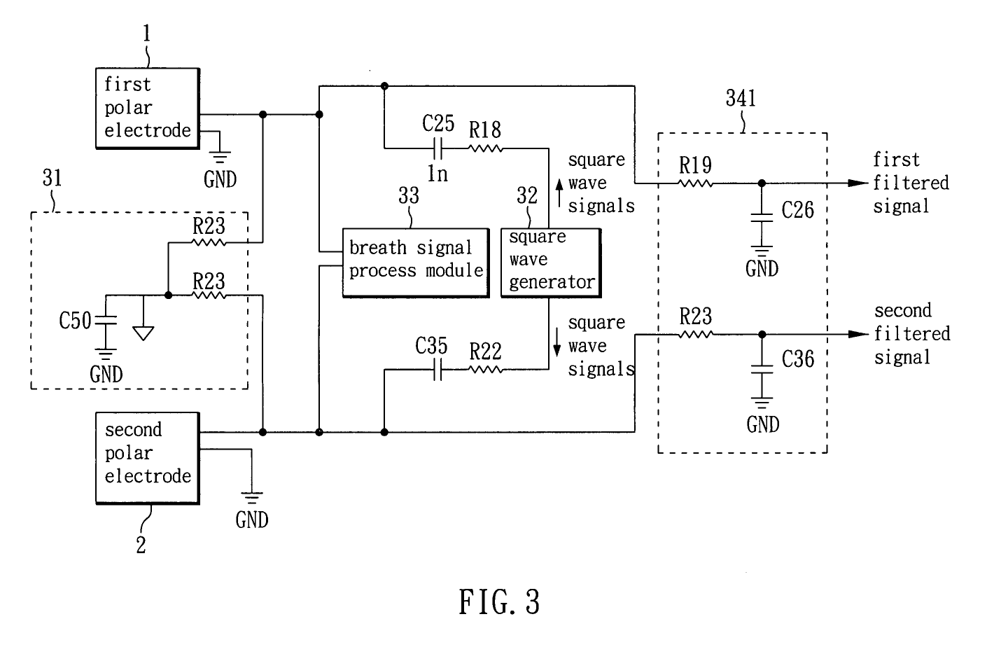

Please refer to FIG. 3. FIG. 3 is a schematic diagram of the peripheral circuit for the two polar electrodes of the system for measuring the ECG and breath signals by using two polar electrodes in accordance with one preferred embodiment of the present invention. The square wave generator 32 generates square wave signals for being imported into the skin of the person under test through the first polar electrode 1 and the second polar electrode 2. The first polar electrode 1 and the second polar electrode 2 receive the first and second measuring signals. The level regulator 31 adjusts the voltage reference of the first and second measuring signals. The breath signal process module 33 and the ECG signal process module 34 proceed to obtain the breath and ECG signals of the person under test.

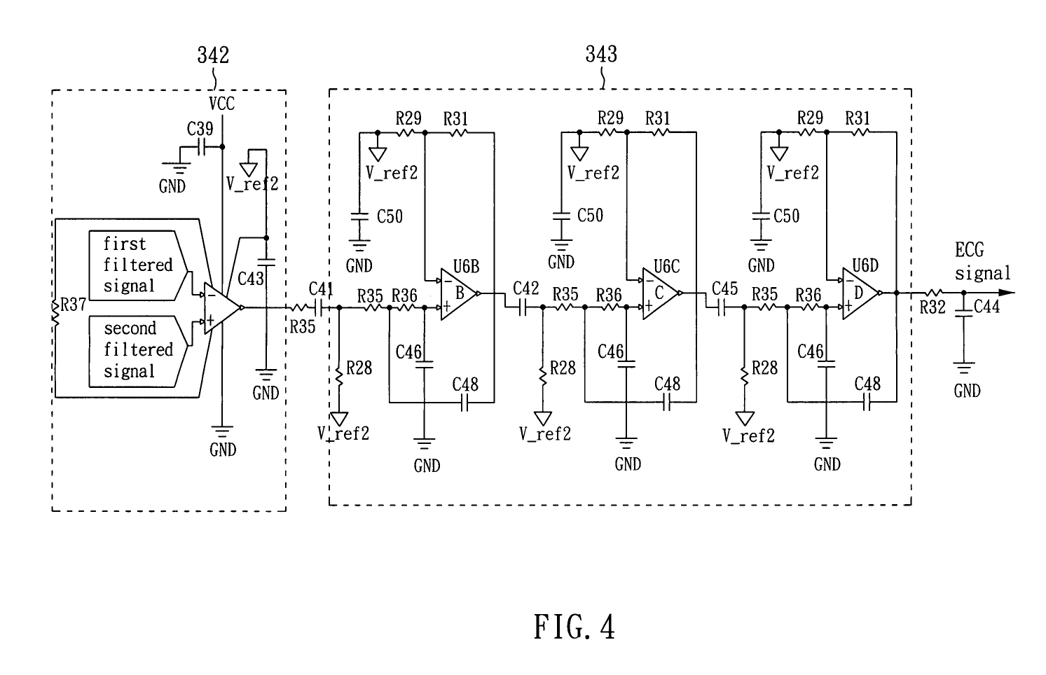

The ECG signal process module 34 receives the first and second measuring signals. First, the filter 341 filters out and rejects the high-frequency signals and noises in the first and second measuring signals. Please refer to FIG. 4. FIG. 4 is a circuit diagram of the ECG signal process module of the system for measuring the ECG and breath signals by using two polar electrodes in accordance with one preferred embodiment of the present invention. The second differential amplifier 342 measures and amplifies the difference between the first and second filtered signals, and then the second filtering amplifier 343 filters out noises in the signals from the second differential amplifier 342 and amplifies the same to generate the ECG signal.

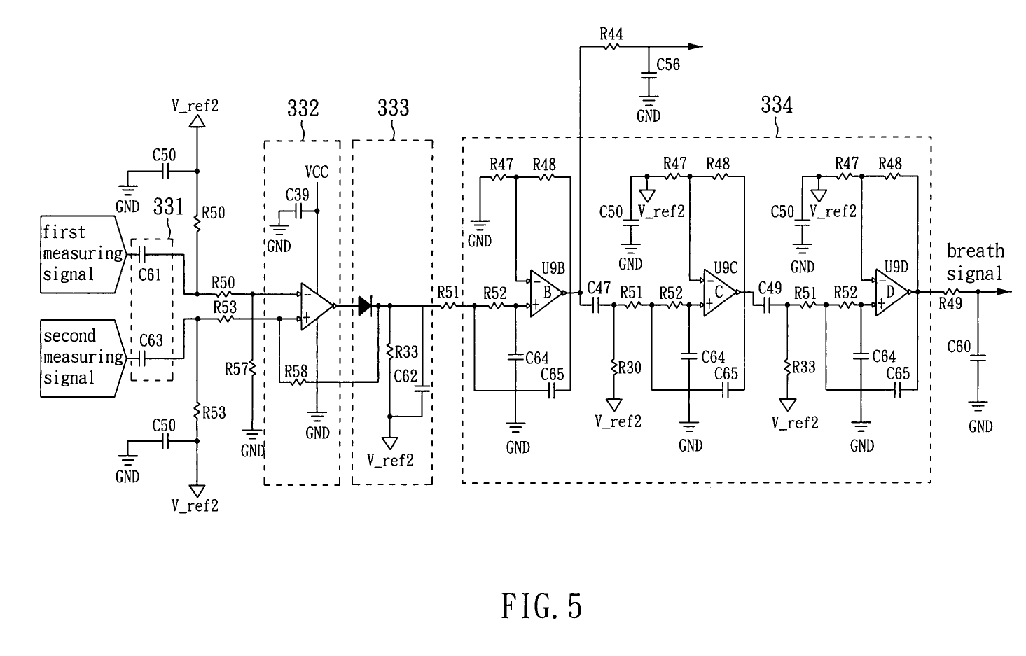

Please refer to FIG. 5. FIG. 5 is a circuit diagram of the breath signal process module 33 of the system for measuring the ECG and breath signals by using two polar electrodes in accordance with one preferred embodiment of the present invention. The signal coupler 331 couples the first and second measuring signals received by the breath signal process module 33. The first differential amplifier 332 measures and amplifies the difference between the first and second measuring signals, and the peak-to-peak detector 333 detects the peak-to-peak value of the signals from the first differential amplifier 332. Finally, the first filtering amplifier 334 filters out noises in the signals from the peak-to-peak detector 333 and amplifies the same to generate the breath signal.

In view of the aforesaid, it is known that only two polar electrodes are attached on the body of the person under test for measuring the ECG and breath signals simultaneously according to the present invention. Therefore, measurement of the ECG and breath signals with the present invention is easier and more comfortable for the person under test.

Although the present invention has been explained in relation to its preferred embodiment, it is to be understood that many other possible modifications and variations can be made without departing from the scope of the invention as hereinafter claimed.

What is claimed is:

1. A system for measuring ECG and breath signals by using two polar electrodes, comprising:
a first polar electrode attached on a first specified part of a person under test for importing electric current and sensing a first measuring signal;
a second polar electrode attached on a second specified part of the person under test for importing electric current and sensing a second measuring signal; and
a breath and ECG signal measuring device connected to the first polar electrode and the second polar electrode, the breath and ECG signal measuring device including a level regulator, a square wave generator, a breath signal process module, and an ECG signal process module, wherein the breath signal process module and the ECG signal process module are connected to the level regulator and the square wave generator respectively, the level regulator adjusts levels of the first and second measuring signals, the square wave generator generates square signals with a fixed frequency, the breath signal process module receives the first and second measuring signals and proceeds to generate a breath signal, the ECG signal process module receives the first and second measuring signals and proceeds to generate an ECG signal.

2. The system as claimed in claim 1, wherein the breath signal process module includes a signal coupler, a first differential amplifier, a peak-to-peak detector and a first filtering amplifier, the signal coupler couples the first and second measuring signals received by the breath signal process module, the first differential amplifier connected to the signal coupler measures and amplifies difference between the first and second measuring signals, the peak-to-peak detector connected to the first differential amplifier detects a peak-to-peak value of signals from the first differential amplifier, the first filtering amplifier connected to the peak-to-peak detector filters out noises in signals from the peak-to-peak detector and amplifying the signals to generate the breath signal.

3. The system as claimed in claim 1, wherein the ECG signal process module includes a filter, a second differential amplifier, and a second filtering amplifier, the filter filters out high-frequency signals and noises in the first and second measuring signals received by the ECG signal process module to generate first and second filtered signals, the second differential amplifier connected to the filtering measures and amplifies difference between the first and second filtered signals, the second filtering amplifier connected to the second differential amplifier filters out noises in signals from the second differential amplifier and amplifies the signals to generate the ECG signal.

4. The system as claimed in claim 1, wherein the first and second specified parts are the left and right hands or beside of the heart of the person under test respectively.

5. The system as claimed in claim 1, wherein the fixed frequency is between 12 KHz and 60 KHz.

6. A method for measuring ECG and breath signals by using two polar electrodes, which is applied in a system including a first polar electrode, a second polar electrode, and a breath and ECG signal measuring device connected to the first and second polar electrodes, the first polar electrode importing electric current and sensing a first measuring signal, the second polar electrode importing electric current and sensing a second measuring signal, the breath and ECG signal measuring device including a level regulator, a square wave generator, a breath signal process module, and an ECG signal process module, the level regulator adjusting levels of signals, the square wave generator generates square signal, the breath signal process module receiving the first and second measuring signals and proceeding to generate a breath signal, the ECG signal process module receiving the first and second measuring signals and proceeding to generate an ECG signal, the method comprises the steps of:
(A) attaching the first and second polar electrodes on a first specified part and a second specified part of a person under test respectively;
(B) the square wave generator generating square wave signals with a fixed frequency for being imported into skin of the person under test through the first and second polar electrodes respectively;
(C) the first and second polar electrodes receiving the first and second measuring signals respectively;
(D) the level regulator adjusting levels of the first and second measuring signals respectively;
(E) the breath signal process module executing for the first and second measuring signals to generate a breath signal;
(F) the ECG signal process module processing the first and second measuring signals to generate an ECG signal; and
(G) outputting the ECG and breath signals.

7. The method as claimed in claim 6, wherein the breath signal process module includes a signal coupler, a first differential amplifier, a peak-to-peak detector, and a first filtering amplifier, in which the signal coupler is connected to the first differential amplifier, the peak-to-peak detector is connected to the first differential amplifier and the first filtering amplifier, and the step (E) comprises the steps of:
(E1) the signal coupler coupling the first and second measuring signals received by the breath signal process module;
(E2) the first differential amplifier measuring and amplifying difference between the first and second measuring signals;
(E3) the peak-to-peak detector detecting a peak-to-peak value of the signals from the first differential amplifier; and
(E4) the first filtering amplifier filtering out and rejecting noises in signal from the peak-to-peak detector, and amplifying the signal to generate the breath signal.

8. The method as claimed in claim 6, wherein the ECG signal process module includes a filter, a second differential amplifier, and a second filtering amplifier, in which the second differential amplifier is connected to the filter and the second filtering amplifier respectively, and the step (F) comprises the steps of:
(F1) the filter filtering out and rejecting high-frequency signals and noises in the first and second measuring signal received by the ECG signal process module to generate first and second filtered signals;
(F2) the second differential amplifier measuring and amplifying difference between the first and second signals; and
(F3) the second filtering amplifier filtering out and rejecting noises in signal from the second differential amplifier and amplifying the signal to generate the ECG signal.

9. The method as claimed in claim 6, wherein the first and second specified parts are the left and right hands or beside of the heart of the person under test respectively.

10. The method as claimed in claim 6, wherein the fixed frequency is between 12 KHz and 60 KHz.
